(19) United States
(12) Patent Application Publication
(10) Pub. No.: US 2024/0343660 A1
(43) Pub. Date: Oct. 17, 2024

(54) IMPROVED SYSTEMS AND PROCESSES FOR HIGH-SELECTIVITY CONVERSION OF MONO-AROMATICS FROM OLEFINS

(71) Applicant: Koch Technology Solutions, LLC, Wichita, KS (US)

(72) Inventors: Brant Lane Aggus, Corpus Christi, TX (US); Timothy Jude Campbell, Ridley Park, PA (US); Daniel Travis Shay, Newtown Square, PA (US)

(21) Appl. No.: 18/294,164

(22) PCT Filed: Jul. 26, 2022
(86) PCT No.: PCT/IB2022/056896

§ 371 Date: Feb. 1, 2024

Related U.S. Application Data
(60) Provisional application No. 63/232,228, filed on Aug. 12, 2021.

(51) Int. Cl. C07C 2/42

(52) U.S. Cl. CPC C07C 2/42; C07C 2529/04

(57) ABSTRACT

The disclosure related to processes for the high-selectivity conversion of olefins to monocyclic aromatic compounds, such as BTX, via the introduction of a weakly coordinating compound to a dehydroaromatization catalyst. Moreover, certain embodiments relate to processes for recycling polyaromatic compounds back to a reactor to improve the yield of said monocyclic aromatic compounds. Moreover, certain embodiments relate to processes for regenerating the dehydroaromatization catalyst.

### RELATED APPLICATIONS

This application claims priority to U.S. Provisional Patent Application No. 63/232,228, filed Aug. 12, 2021, the entirety of which is incorporated herein by reference.

### BACKGROUND

The present disclosure relates to systems and processes for the high-selectivity conversion of olefins into monocyclic aromatic compounds using a dehydroaromatization catalyst. The resulting monocyclic aromatic compounds include BTX (mixtures of benzene, toluene, and xylene isomers) which are important chemicals in the petroleum refining and petrochemical industries. In order to achieve high-selectivity conversion of olefins into monocyclic aromatic compounds, temperatures greater than 450° C. are required. However, these high temperatures result in detrimental effects to the catalyst (i.e. catalyst deactivation via coke formation on the catalyst surface and inside the pores blocking active sites). In addition, the formation of polyaromatic compounds (e.g., methyl- and dimethylnaphthalenes) can reduce the efficiency of the process. Accordingly, there is a need in the art for processes that provide high-selectivity conversion of olefins to monocyclic aromatic compounds under conditions that do not cause premature catalyst deactivation.

### SUMMARY

The embodiments disclosed herein provide an improved monocyclic aromatic compounds production. Disclosed herein are systems and methods for the high-selectivity (i.e., a yield greater than 25%, more preferably greater than 45%, and most preferably greater than 65%) conversion of olefins to monocyclic aromatic compounds. The processes described herein addresses a problem recognized in the art with respect to the high severity operation necessary for the high-selectivity conversion of olefins to monocyclic aromatic compounds, such as detrimental effects to the catalyst (i.e. catalyst deactivation via carbon formation on the catalyst surface and inside the pores blocking active sites).

Embodiments herein seek to overcome these detrimental effects through the addition of a weakly coordinating compound which mitigates the rate of carbon formation on the catalyst surface by attenuating surface acidity (thereby limiting non-selective product formation). A complimentary means to improve monocyclic aromatic compounds yield is through recycling of polyaromatic products of the process to a catalyst or gasifier or pyrolysis reactor which can crack/dehydroaromatize them to a mixture of monocyclic aromatic compounds and light gas. The addition of a weakly coordinating compound in combination with the recycling of polyaromatic products confers high-selectivity conversion on the reaction. In addition, the high-selectivity conversion is further improved by higher temperatures and pressures, removal of gas contaminants, and/or regeneration of the catalyst.

In a first aspect provided herein, a process for high-selectivity conversion of a first composition comprising olefins to a second composition comprising at least one monocyclic aromatic compound, includes the steps of:
a) introducing the first composition to at least one catalyst capable of converting olefins to at least one monocyclic aromatic compound;
b) operating said process using a reactor at high severity to result in the production of at least one monocyclic aromatic compound; and at least one of:
c1) introducing at least one weakly coordinating compound to the catalyst; and
c2) forming at least one polyaromatic compound in said process, wherein said at least one polyaromatic compound is recycled to a gasifier or pyrolysis reactor or catalytic reactor.

In an embodiment of the process of the first aspect discussed herein, the process further comprises the step of subjecting the first composition with a nitrogen and/or sulfur atom and/or halogen-containing compound removal process. Further, in an embodiment of the process of the first aspect discussed herein, said compound removal process is an adsorbing guard bed, a catalytic process, or a solvent-based absorption process.

In an embodiment of the process of the first aspect discussed herein, said at least one catalyst is porous aluminosilicate or zeolitic material with a portion of its pores in the micro-, or meso-, and/or macro-range.

In an embodiment of the process of the first aspect discussed herein, said at least one catalyst is subject to a regeneration process, wherein said regeneration process comprises the introduction of inert gas and/or an oxidant and/or a reductive fluid.

In an embodiment of the process of the first aspect discussed herein, said high severity is in an outlet or inlet of said reactor, or intra-reactor, and further wherein said high severity is >425° C.; more preferably >450° C., and most preferably >475° C.

In an embodiment of the process of the first aspect discussed herein, said weakly-coordinating compound reduces the H—O bond frequency of zeolite or aluminosilicate framework by about 1 to 300 cm⁼¹ as measured by FT-IR.

In an embodiment of the process of the first aspect discussed herein, said olefins have a carbon chain length of C₂ to C₄.

In an embodiment of the process of the first aspect discussed herein, said olefins are from direct or indirect gasification processes. Further, in an embodiment of the process of the first aspect discussed herein, said gasification processes are coal/petroleum-based, biomass-based, waste plastic, municipal solid waste, refuse-derived fuel, mixed plastic or other waste sources.

In an embodiment of the process of the first aspect discussed herein, the process further includes the step of removing at least one by-product before introducing the first composition to at least one zeolitic catalyst. Further, in an embodiment of the process of the first aspect discussed herein including the step of removing at least one by-product, said at least one by-product is removed by adsorption, aqueous redox reaction, solvent or solid absorption, electrostatic precipitation, centrifugal separation, or filtration.

In an embodiment of the process of the first aspect discussed herein, the reactor is a fixed bed, fluidized bed, or moving bed.

In an embodiment of the process of the first aspect discussed herein, said process is operated at a pressure of 50 psi to about 500 psi.

In an embodiment of the process of the first aspect discussed herein, said high-selectivity conversion of a first composition comprising olefins to a second composition comprising the monocyclic aromatic compounds is >25%, more preferably >45%, and most preferably >65%.

In an embodiment of the process of the first aspect discussed herein, said weakly-coordinating compound is carbon monoxide.

In an embodiment of the process of the first aspect discussed herein, the process further includes: subjecting a feed gas to a compound removal process to yield the first composition, the compound removal process including: inputting the feed gas and a compound removal solution into a compound removal contactor; and outputting from the reactor an output including the first composition. Further, in an embodiment of the process of the first aspect discussed herein, the compound removal solution including circulating ammonium polysulfide (APS). Further, in an embodiment of the process of the first aspect discussed herein, the compound removal solution including diammonium polysulfide. Further, in an embodiment of the process of the first aspect discussed herein the compound removal solution further includes (in addition to APS or diammonium polysulfide) ammonium hydroxide.

In an embodiment of the process of the first aspect discussed herein, the compound removal solution includes a mixture of a first compound removal solution component and a second compound removal solution component in aqueous solution at a weight ratio of 1:2 at approximately 20-40° C. Optionally, the second compound removal solution component is an alkaline solution.

In second aspect, a process for supplying a first composition to a downstream catalytic process, includes: inputting the feed gas and a compound removal solution into a compound removal contactor; and operating the reactor to remove at least one by-product from the feed gas; and, outputting from the reactor an output composition to the downstream catalytic process, the output composition including at least one olefin.

In an embodiment of the process of the second aspect discussed herein, the compound removal solution including circulating ammonium polysulfide (APS).

In an embodiment of the process of the second aspect discussed herein, the compound removal solution including diammonium polysulfide.

In an embodiment of the process of the second aspect discussed herein, the compound removal solution further includes ammonium hydroxide.

In an embodiment of the process of the second aspect discussed herein, the compound removal solution includes a mixture of a first compound removal solution component and a second compound removal solution component in aqueous solution at a weight ratio of 1:2 at approximately 20-40° C. Optionally, the second compound removal solution component is an alkaline solution.

In an embodiment of the process of the second aspect discussed herein, the process further comprises: outputting from the compound removal contactor an additional output including a by-product removal output; and stripping at least one component of the by-product removal output in a vessel separate from the reactor.

In an embodiment of the process of the second aspect discussed herein, the by-product removal output including ammonium polysulfide (APS).

In an embodiment of the process of the second aspect discussed herein, the at least one component of the by-product removal output includes gaseous ammonia and hydrogen sulfide.

In an embodiment of the process of the second aspect discussed herein, the process further includes recycling the at least one component to an upstream process.

In an embodiment of the process of the second aspect discussed herein, the process further includes recycling at least one additional component of the by-product to the upstream process.

In an embodiment of the process of the second aspect discussed herein, the at least one component includes ammonium polysulfide (APS), and the process further includes circulating the APS to the compound removal contactor.

### BRIEF DESCRIPTION OF THE DRAWINGS

FIG. 1 shows a schematic of an exemplary process for high-selectivity conversion of a first composition comprising olefins to a second composition comprising at least one monocyclic aromatic compound, in embodiments.

FIG. 2 shows the impact a few of certain by-products of gasification have on the reduction of the cycle time of a zeolite dehydroaromatization catalyst.

FIG. 3 shows an embodiment of the process of FIG. 1, including a regeneration process 300, in embodiments

FIG. 4 shows one example of careful introduction of an oxidant and slowly increasing temperature allowing for complete removal of coke formed on a deactivated catalyst.

FIG. 5 is a schematic of an exemplary process for high-selectivity conversion of a first composition comprising olefins to a second composition comprising monocyclic aromatics including utilizing a recycle stream to improve monocyclic aromatic compounds yield, in embodiments.

FIG. 6 shows an example schematic 600 of a method for hydrogen cyanide removal from a feed stream using ammonium polysulfide contacting scheme, in embodiments.

FIG. 7 is a graph depicting the effect of a carbon monoxide feed on catalyst lifetime.

### DETAILED DESCRIPTION

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. In case of conflict, the present specification, including definitions, will control.

In addition, the materials, methods, and examples are illustrative only and not intended to be limiting. The details of one or more embodiments of the invention are set forth in the accompanying drawings and the description below. Other features, objects, and advantages of the invention will be apparent from the description and the drawings, and the claims. The word “comprising” in the claims may be replaced by “consisting essentially of” or with “consisting of,” according to standard practice in patent law.

Like reference numerals within the figures are intended to refer to the same components, even if in different figures. Thus, even if in different figures, the discussion of one element referenced by a given reference numeral in one figure applies equally to the other figure containing said same given reference numeral, unless otherwise specified.

The process flow diagrams are provided, herein, as illustrations of the general process. Certain derivations are known to those skilled-in-the-art, such as further integration with conventional feed separation and fractionation schemes, various heat integration options and product stabilization schemes.

Unless specifically stated otherwise or obvious from context, as used herein, the term “about” and “approximately” are understood as within a range of normal tolerances in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.” In any of the embodiments herein, the monocyclic aromatic compounds may include BTX (mixtures of benzene, toluene, and xylene isomers). In any of the embodiments herein, the monocyclic aromatic compounds may include ethylbenzene, and the mixture may then be referred to as BTEX. In some embodiments, xylene isomers comprise o-xylene, m-xylene, p-xylene, or combinations thereof.

A “catalyst” refers to a dehydroaromatization catalyst utilized for the conversion of olefins into monocyclic aromatic compounds. In an embodiment, the catalyst is porous aluminosilicate or zeolitic material with a portion of its pores in the micro-, meso- and/or macro-range. In some embodiments, the catalyst is a zeolite catalyst having a pentasil structure. In some embodiments, the zeolite catalyst is ZSM-5 or ZSM-11. In some embodiments, the zeolite is a ZSM-5 MFI zeolite. The Si02:Al03 ratio of the zeolite may vary between 20 and 50. Zeolites such as ZSM-5 are capable of converting ethylene and other unsaturated hydrocarbons into monocyclic aromatic compounds such as benzene via a complex sequence of oligomerization, isomerization, cracking and cyclization reactions that are believed to initiate on Bronsted acid sites of the zeolite. The catalyst may be promoted or unpromoted. Promoting catalyst is known in the art, and also referred to as loading. This is a well-known procedure which typically involves impregnating or ion-exchanging the catalyst with soluble salts of the promoting elements. In another embodiment, the catalyst is a heterogeneous catalyst comprising aluminosilicate in the range between about 1% to about 99%, amorphous silica, amorphous alumina, or a combination thereof, in a range of about 0% to about 99%. In further embodiments, the aluminosilicate contains at least 10% of its total porosity having a mean pore diameter of less than 20 nm. In further embodiments, the catalyst has a total surface area of at least 90 m²/gram. In an embodiment, at least one catalyst is porous aluminosilicate or zeolitic material with a portion of its pores in the micro-, meso-, and/or macro-range. In an embodiment, at least one catalyst is subject to a regeneration process, wherein said regeneration process comprises the introduction of inert gas and/or an oxidant and/or a reductive fluid at elevated temperature.

As used herein, the “weakly-coordinating compound” reduces the H—O bond frequency of the zeolite catalyst or aluminosilicate catalyst framework by about 1 to 300 cm⁼¹ as measured by FT-IR. In any embodiments herein, the weakly coordinating compound may be a Lewis base. In any embodiments herein, the weakly coordinating compound may be a labile compound. Non-limiting examples of weakly coordinating compounds include carbon monoxide, acetonitrile, dimethylsulfoxide, tetrahydrofuran, acetone, pyridine or tetrahydrothiophene.

As used herein, “high severity operation” or “high severity conditions” refer to temperatures within a reactor (e.g., at the input of the reactor, output of the reactor, or intra-reactor) within a range of about 400° C. to about 500° C. In some embodiments, the high severity conditions are characterized by a temperature above 425° C. In some embodiments, the high thermal severity is characterized by a temperature above 450° C. In some embodiments, the high thermal severity is characterized by a temperature above 475° C. In some embodiments, the high thermal severity is characterized by a temperature in the range of about 450° C. to about 500° C. In some embodiments, “high severity operation” or “high severity conditions” alternatively or additionally includes pressure within a range of about 50 psi to about 500 psi. In embodiments, “high severity operation” or “high severity conditions” alternatively or additionally includes weight hour space velocity (WHSV) between about 0.01 and 10/hour. High severity operation at low pressure impacts olefin conversion due to the Langmuir Adsorption Isotherm (LAI). In particular, the LAI states the adsorption of a molecule on a catalyst surface is proportional to the pressure/temperature. As such, as temperature increases, adsorption decreases and to overcome this, pressure must increase. Thus, to increase conversion yield of monocyclic aromatic compound per converted olefin, the severity within the reactor must be increased. However, this increase results in lower cycle times of the catalyst in the reactor. Accordingly, the present application provides an improvement by adding a weakly coordinating base to the reaction so that the cycle time of the catalyst increases.

In the present disclosure, it was unexpectedly found that one way to prevent premature catalyst deactivation due to coke formation on the catalyst surface when operating at temperatures that favor high-selectivity of monocyclic aromatic compounds from olefins, i.e., above 400° C., is to add a weakly coordinating compound to the feed. In theory, a weakly coordinating compound will coordinate with the most acidic sites of the catalyst and inhibit formation of the coke precursors, thus extending the cycle time of the catalyst. One example of a weakly coordinating compound is carbon monoxide. In some embodiments, the carbon monoxide is provided in an amount ranging from 0.1% by weight to 25% by weight. In some embodiments, the carbon monoxide is provided in an amount ranging from 0.1% by weight to 10% by weight. In some embodiments, the carbon monoxide is provided in an amount ranging from 3% by weight to 7% by weight. In a preferred embodiment, the carbon monoxide is provided in an amount ranging from 5% by weight to 7% by weight.

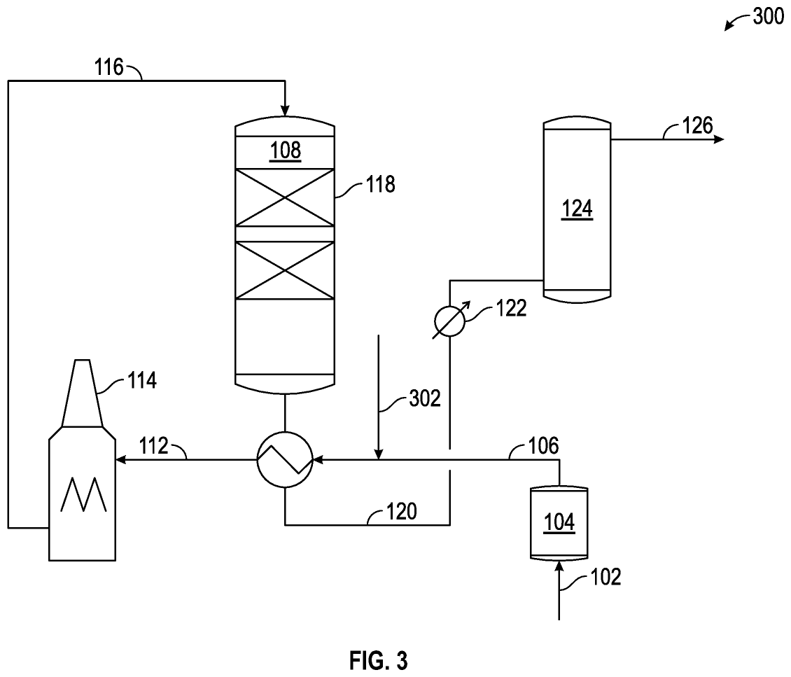

When added to the feed in these given amounts, carbon monoxide can prolong the cycle time of the catalyst by about 15%, (see, e.g. FIG. 3).

A complimentary means to improve the high-selectivity of monocyclic aromatic compounds from olefins is to recycle polyaromatic products of the processes described herein to a catalytic reactor, gasifier, or pyrolysis reactor which can crack or dehydroaromatize them to a mixture of monocyclic aromatic compounds and a composition comprising a light (e.g., C1 to C4 carbon chain length) hydrocarbon mixture with a 37° C. (or between 20-40° C.) vapor pressure range from 2 to 51 psia. Under high-severity conditions, the catalyst could produce as much as 15 wt % polyaromatic products, such as methyl- or dimethylnaphthalenes. Recycling these polyaromatic products back to a catalyst, or gasifier, or pyrolysis reactor can improve the monocyclic aromatic compound yield of the process.

Accordingly, in an aspect, the disclosure provides a process for the high-selective conversion of olefins to monocyclic aromatic compounds, comprising the steps of:
a) introducing the first composition to at least one catalyst capable of converting olefins to at least one monocyclic aromatic compound;
b) operating said process using a reactor at high severity to result in the production of at least one monocyclic aromatic compound; and at least one of:
c1) introducing at least one weakly coordinating compound to the catalyst; and
c2) forming at least one polyaromatic compound in said process, wherein said at least one polyaromatic compound is recycled to a gasifier or pyrolysis reactor or catalytic reactor.

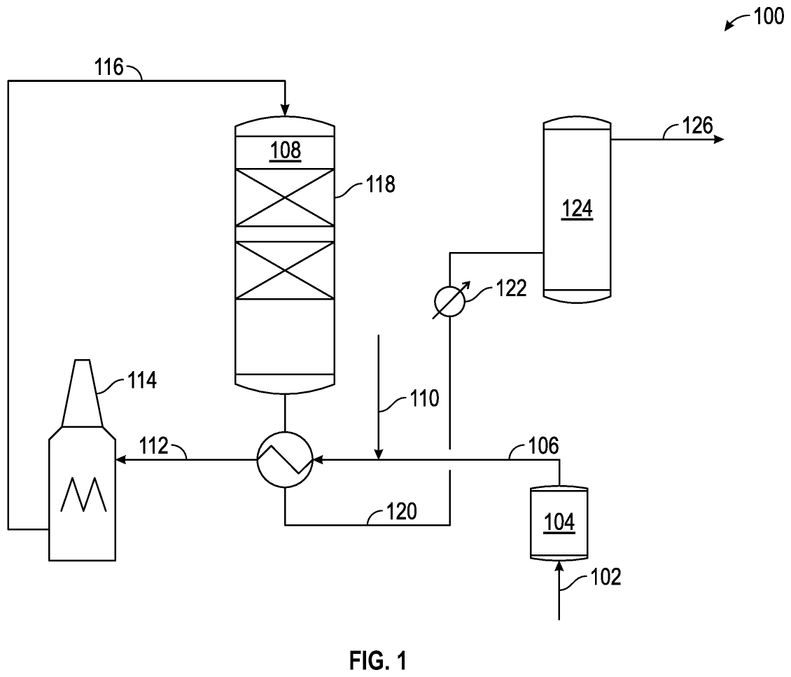

FIG. 1 depicts an exemplary schematic 100 for the high-selectivity conversion of a first composition comprising olefins to a second composition comprising monocyclic aromatic compounds. A gas feed containing a first composition of olefins enters the process as stream 102 and may include a gas feed from an up-stream process, such as a catalytic conversion process. In some embodiments the olefins are from stream 102 in the form of a fluidized catalytic cracking off-gas feed, residue FCC (RFCC) off-gas feed, or coker off-gas feed, or other refinery streams. In some embodiments, the olefins in stream 102 are olefins with a C2 to C4 carbon chain length. In some embodiments, the olefins in stream 102 are from a direct or indirect gasification process upstream from schematic 100. In an embodiment, the gasification processes are coal/petroleum-based, or biomass-based. In a further embodiment, the gasification process is waste-based and comprises black liquor originating from paper pulp.

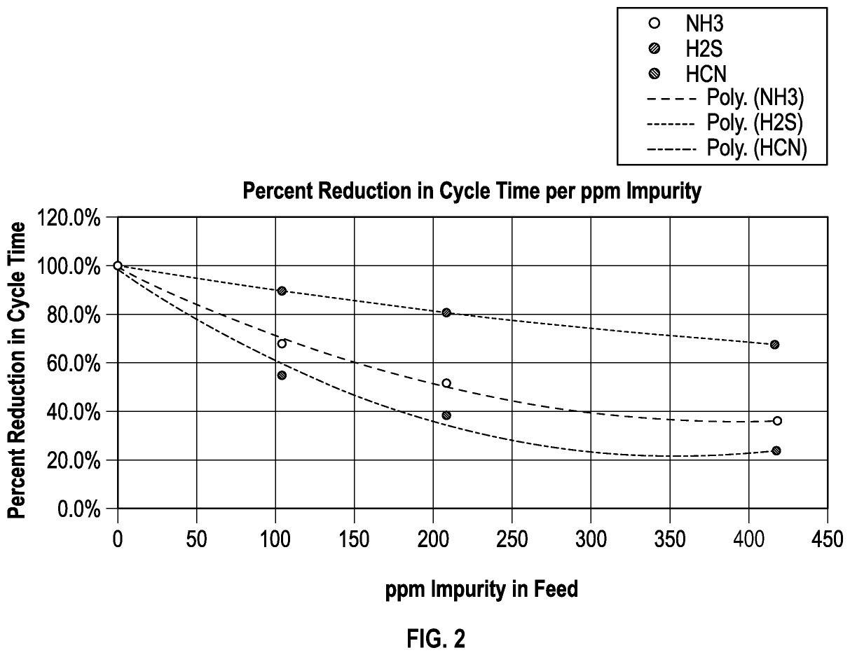

Certain up-stream processes that produce olefins suitable for use as stream 102 in the presently disclosed processes also produce by-products containing sulfur, nitrogen, or chlorine and the like. When mixtures of these olefins and by-products are used as feedstock for catalysts 118 (and in some embodiments, zeolite catalysts) in the reactor 108, as discussed below, the by-products can cause premature deactivation of the catalyst due to selective poisoning of the active sites. In instances where a waste gasification process produces a variety of donor molecule by-products along with olefins and synthesis gas, feeding said mixture to a catalyst operating under high severity conditions results in rapid deactivation of the catalyst if the catalyst is left untreated. FIG. 2 shows the impact that common by-products of gasification have on reduction of the cycle time of a zeolite catalyst, in embodiments. Treating these impurities is key to economic operation of zeolite catalysts in instances where olefin sources contain a donor by-product molecule, e.g. hydrogen cyanide (HCN), ammonia (NH₃), hydrogen sulfide (H₂S). Accordingly, in some embodiments, the process subjects stream 102 a compound removal process 104 resulting in treated stream 106. Compound removal process(es) 104 are optional.

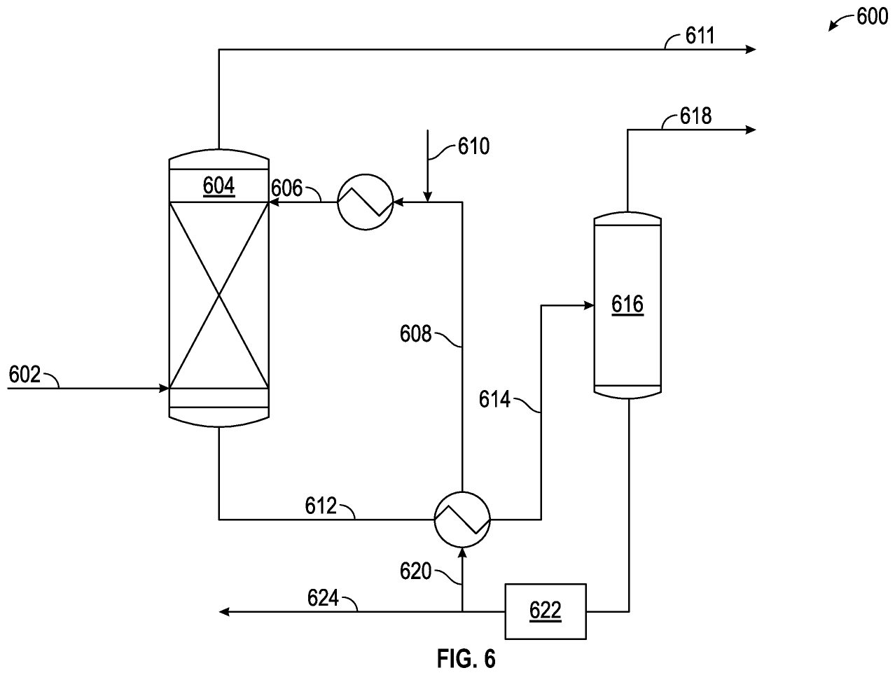

The compound removal process 104 may remove nitrogen-, sulfur-, or halogen-containing compound or other undesired compounds from the stream 102. In some embodiments, the removal process 104 utilizes an adsorbing guard bed, a catalytic process, a solvent-based or solid-based absorption process, an adsorption process, an aqueous redox reaction, electrostatic precipitation, centrifugal separation, a filtration processes, neutral pH water or alkaline solution wash, or any combination thereof. One example of the compound removal processes 104 is nitrogen components are removed using a vessel containing solid adsorbent. The compound removal process(es) 104 may include subjecting the first composition (stream 102) to a nitrogen and/or sulfur atom and/or halogen-containing compound removal process. Another example of compound removal process(es) 104 is discussed below with reference to FIG. 6.

The resulting treated stream 106 (or input stream 102 where removal process 104 is not included) is sent through exchangers and heaters prior to reaction. Prior to entry to a reactor 108, an additive stream 110 introduces a second composition of a weakly coordinating compound to the treated stream 106 (or stream 102) prior to reaction resulting in a combined stream 112 that is input into reactor 108. In an embodiment, the reactor 108 is a fixed bed, fluidized bed, or moving bed. In an embodiment, the reactor 108 is a catalytic reactor, a gasifier, or a pyrolysis reactor.

In embodiments, the combined stream 112 may be heated by a fired heater 114 prior to entry into reactor 108 resulting in a hot feed 116. The hot gas feed 116 enters reactor 108 and is reacted over the catalyst 118 bed(s) containing a dehydroaromatization catalyst. In an embodiment, the second composition of a weakly coordinating compound introduced via additive stream reduces the H—O bond frequency of the catalyst 118 (e.g., a zeolite or aluminosilicate framework) by about 1 to 300 cm⁻¹ as measured by FT-IR.

After the compounds react, the resulting product stream is output from the reactor 108 as output stream 120 and is cooled using cooler 122. Cooler 122 is optional. The output stream 120, containing 2-phase liquid and vapor products are separated in vessel 124. The resulting products, exit as a separated product stream 126.

Although shown as being added to the treated stream 106 (or stream 102), the additive stream 110 may be introduced at any point prior to entry of the hot gas feed 116 into the reactor 108. In one embodiment, the additive stream 110 may be introduced to treated stream 106 or stream 102 after treated stream 106 or stream 102 are heated by fired heater 114. Furthermore, the additive stream 110 may be introduced directly to the reactor 108 as a separate stream than the hot feed 116 without departing from the scope hereof.

In an embodiment, reactor 108, during conversion of a first composition comprising olefins to a second composition comprising monocyclic aromatic compounds, is subjected to high severity. In an embodiment, the high severity is in an outlet or inlet of said reactor 108, or intra-reactor. In an additional or alternative embodiment, the high severity is >450° C.; more preferably >500° C., and most preferably >550° C.

In an embodiment, reactor 108, during conversion of a first composition comprising olefins to a second composition comprising monocyclic aromatic compounds, is subjected to a pressure of 50 psi to about 500 psi.

In an embodiment, the high-selectivity conversion of a first composition comprising olefins to a second composition comprising monocyclic aromatic compounds achieved via the process shown in schematic 100 is >25%, more preferably >45%, and most preferably >65%.

### Catalyst Regeneration:

FIG. 3 shows an embodiment of the process of FIG. 1, including a regeneration process 300, in embodiments. In an embodiment, the catalyst 118 is subject to a regeneration process, wherein said regeneration process comprises the introduction of regeneration compound 302 and operating the reactor 108 at a regeneration temperature. In one example, the regeneration compound 302 is one or more of an inert gas, an oxidant, a reductive fluid, and any combination thereof. In an embodiment, operating the reactor 108 at a regeneration temperature includes gradually increasing the temperature in the reactor 108 to >450° C. over a regeneration period. In some embodiments, the temperature of reactor 108 during regeneration process is increased to between 300° C. and 700° C. during the course of the regeneration process. In some embodiments, the temperature is increased to about 500° C. In some embodiments, the oxidant is provided at low concentrations (e.g., about 1% oxygen gas in nitrogen gas) and increased over the course of the regeneration process.

Thus, in instances of catalysts with a high degree of coke formed in/on the catalyst, rapid introduction of an oxidant may cause an excessive exotherm which can damage the catalyst. In these instances, it may be favorable to slowly introduce either an inert gas or oxidant at lower temperatures and increase temperature accordingly to maintain temperatures favorable for catalyst regeneration but not too severe to negatively impact catalyst structure. Accordingly, the above temperature ranges and concentration of the introduced regeneration compound 302 may be based on the amount of coke formation, catalyst type, and other factors that relate to catalyst damage during the regeneration thereof.

Rapid deactivation of zeolite catalysts (either by coke formation due to high severity operation or impurity poisoning) is economically unfavorable if the catalyst is not or cannot be regenerated. In high severity operation for high-selectivity conversion of monocyclic aromatic compounds from olefins, as much as 20 to 25 wt % carbon can accumulate on the surface and inside the pores of the catalyst upon deactivation. Additionally, impurities such as HCN or NH₃ will coordinate with the active sites of the catalyst causing further deactivation. Regeneration of the catalyst and removal of both the coke formed in/on the zeolite catalyst and donor impurities such as NH₃ or HCN can be accomplished through the use of an oxidant e.g. air, N₂/O₂ or H₂O₂ and allowing for extended catalyst lifetime.

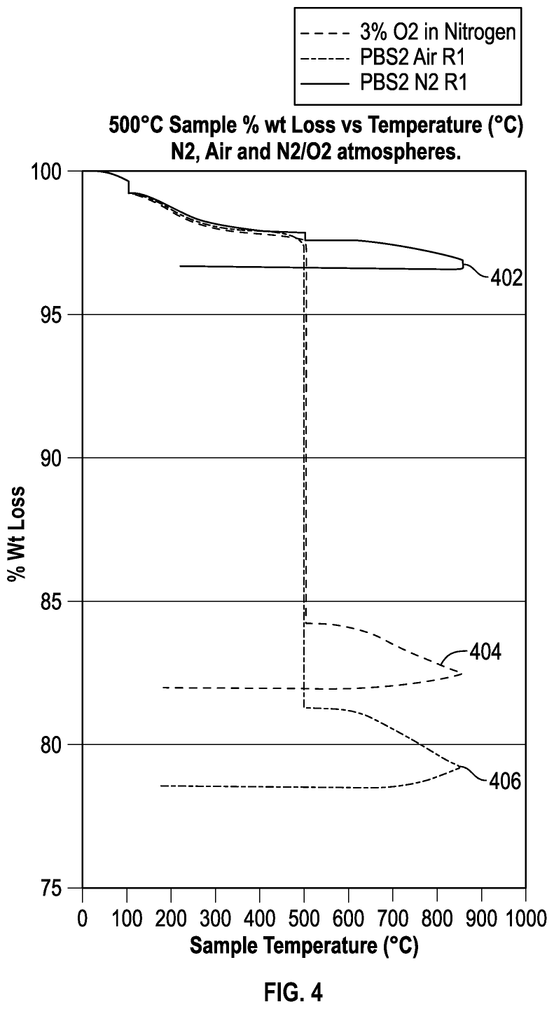

FIG. 4 shows one example of careful introduction of an oxidant and slowly increasing temperature allowing for complete removal of coke formed on a deactivated catalyst. Zeolite catalyst that was deactivated operating in high-severity mode was regenerated using nitrogen (402), 3% oxygen in nitrogen (404) and air (406) showing minimal, modest, and complete coke removal from the catalyst respectively.

### Recycling of Polyaromatic Compounds

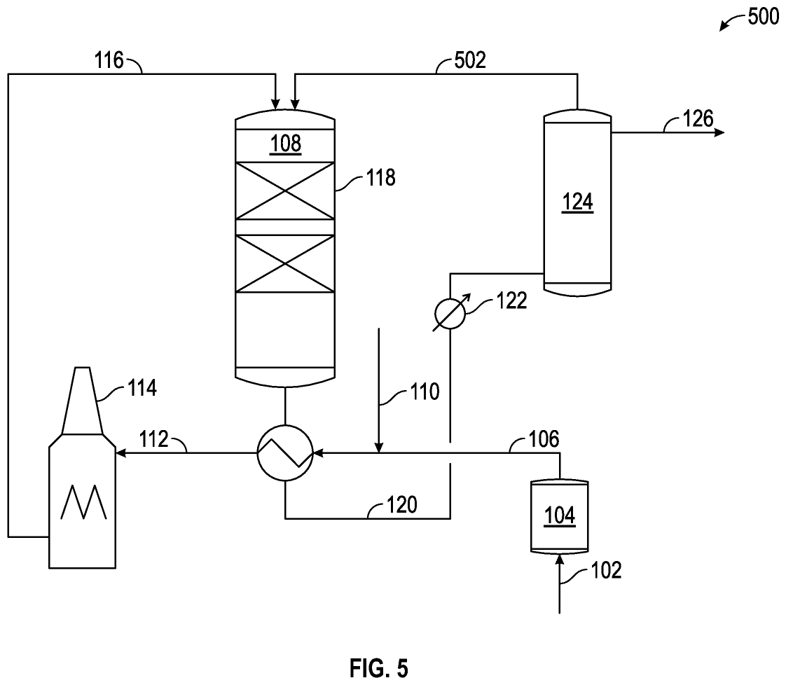

FIG. 5 depicts an exemplary schematic 500 for high-selectivity conversion of a first composition comprising olefins to a second composition comprising monocyclic aromatic compounds including utilizing a recycle stream to improve aromatics yield, as shown in certain embodiments. In some embodiments, during high severity operation, process 100 could produce as much as 15 wt % polyaromatic compounds, such as methyl- or dimethylnaphthalenes. Accordingly, a recycle stream 502 may be input back into reactor 108 (e.g., from separator vessel 124, or as a separate output from the reactor 108). Recycle stream 502 may include polyaromatic products, such as methyl- or dimethylnaphthalenes. Use of recycle stream 502 back to the catalytic reactor, gasifier, or pyrolysis reactor can improve the monocyclic aromatic compounds yield of the process and favorable economic operation.

### Compound Removal Process Embodiments:

The below discussion relates to examples of the compound removal process 104 discussed above with respects to FIGS. 1-5. These examples are intended to be non-limiting. Other types of compound removal processes may be implemented as discussed above with regards to compound removal process 104. Moreover, two or more of the compound removal processes discussed herein (and other types of compound removal processes) may be used in combination with each other. One of the multiple compound removal processes may remove a first one or more compound, and another of the multiple compound removal processes may remove a second one or more compound.

The production of olefins and synthesis gas during the process of waste gasification produces a variety of donor molecule by-products. When fed to a catalyst operating in high-severity mode to favor the production of monocyclic aromatic compounds, a rapid deactivation of the catalyst will occur if the donor molecule by-products are left untreated. These impurities comprise halogens, sulfur-containing compounds, and nitrogen-containing impurities (See FIG. 2). Treating these impurities significantly improves to the economic operation of zeolite catalysts in instances where olefin sources contain donor by-product molecules such as halogens, sulfur-containing compounds, and nitrogen-containing compounds.

As used herein, a “donor molecule by-product” or “common by-product” is a gasifier impurity. Non-limiting examples of these molecules include HCN, NH₃, or H₂S. These compounds can cause pre-mature deactivation due to selective poisoning of catalytic active sites and can be removed by adsorption, catalytic conversion, or a homogenous reaction.

In one example of compound removal process 104, contact of the feed gas (e.g., stream 102) with a neutral pH water wash or preferentially an alkaline solution (sodium hydroxide, potassium hydroxide, or ammonium hydroxide) can be utilized to remove halogen and some sulfur-containing compounds. In one example of compound removal process 104, contact of the feed gas (e.g., stream 102) with an acid solution can remove some nitrogen-containing compounds. Reacting some nitrogen-containing compounds with ammonium polysulfide solution by contacting them in a liquid absorber system can reduce them to an acceptable level in the feed to the aromatization catalyst bed. Fixed-bed alumina absorbent can be utilized to remove many of the contaminants listed above, particularly basic nitrogen and halogens.

FIG. 6 shows an example schematic 600 of a method for hydrogen cyanide removal from a feed stream using ammonium polysulfide contacting scheme, in embodiments.

Feed gas 602 is input into a compound removal contactor 604. Feed gas 602 is an example of stream 102 of FIG. 1, and thus the discussion of stream 102 above applies equally as well to feed gas 602. The compound removal reactor 604 also includes an input of a compound removal solution 606. In an embodiment, the compound removal solution 606 is circulating a solution of circulating ammonium polysulfide solution. In an embodiment, the compound removal solution 606 includes, a mixture of ammonium polysulfide (APS) and ammonium hydroxide. In an embodiment, the compound removal solution 606 includes, a mixture of diammonium polysulfide and ammonium hydroxide. In an embodiment, the compound removal solution 606 may include a circulating first compound removal solution (or compound) component 608 combined with a second compound removal solution (or compound) 610. In an embodiment, the first compound removal solution (or compound) component 608 is circulating ammonium polysulfide. In an embodiment, the first compound removal solution (or compound) component 608 is circulating diammonium polysulfide. In an embodiment, the second compound removal solution (or compound) component 610 is an alkaline component (e.g., any one or more of sodium hydroxide, potassium hydroxide, or ammonium hydroxide). In an embodiment, the second compound removal solution (or compound) component 610 is ammonium hydroxide. In an embodiment, the mixture of the first compound removal solution (or compound) component 608 and the second compound removal solution (or compound) component 610 may be in aqueous solution at a weight ratio of 1:2, at approximately 20-40° C.

Hydrogen cyanide in the feed gas 602 reacts in compound removal contactor 604 with ammonium polysulfide in the compound removal solution 606 to form output composition 611 and by-product removal output 612 from the reactor including ammonium thiocyanate and hydrogen sulfide through the following mechanism:

2 HCN+(NH₄)Sₓ→2 NH₄SCN+H₂S+Sₓ

The output composition 611 is analogous to the treated stream 106 in FIG. 1.

This by-product removal output 612 is then input 614 into a second vessel 616 where stripping of the rich ammonium polysulfide (APS) solution in the output 612 occurs. In embodiment, the stripping of the rich ammonium polysulfide (APS) solution occurs at a temperature of 80-110° C. in the vessel 616 resulting in an output 618 of gaseous ammonia and hydrogen sulfide. This output 618 may be recycled back to the upstream waste gasification process for removal by acid and caustic scrubbing, or a sulfur recovery unit for further processing. A second output 620 may be exchanged back to be included in the circulating first component 608. Prior to recirculation, the APS may pass through filter or settling vessel 622 in which sulfur in the rich APS solution may be removed by settling or filtration. Additionally, a stream 624 including at least part of the remaining rich APS solution may be recycled back to the upstream waste gasification process.

In an embodiment, HCN in the feed gas 602 can be catalytically hydrolyzed in vessel 604, which can be a fixed bed reactor in this case. This can be carried out by contacting the gas with a metal oxide catalyst, preferably zinc oxide, at temperature greater than 100° C. Product NaH₃ from the hydrolysis reaction can be absorbed in a subsequent water wash column.

### EXAMPLES

### Example 1

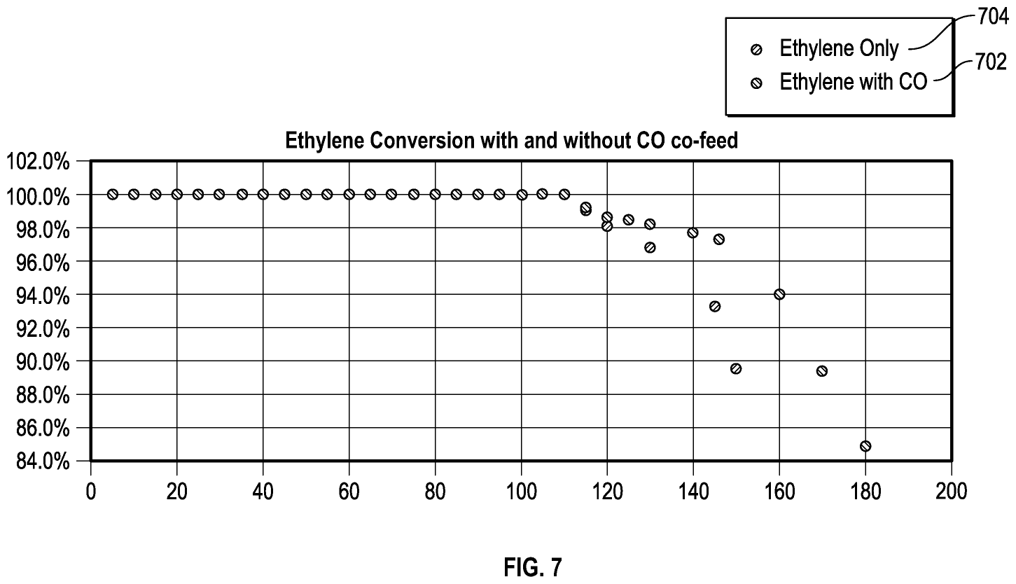

Zn/Ln modified ZSM5 catalyst was used to effect olefin conversion of 17 wt % ethylene in N₂ as stream 102, to BTX as aromatics stream 122 with (702) and without (704) a weakly coordinating compound 114 added (e.g., 5 wt % CO feed). The conversion of ethylene to BTX over time under both conditions is shown in FIG. 7. Notably, addition of CO prolonged the lifetime (e.g., the cycle time) of the catalyst an additional 20 hours (a 13% increase in catalyst efficiency).

### Example 2

5 grams of 16 mesh Zn/Ln modified ZSM5 catalyst charged in a 0.500 inch od×0.035 inch wall 316SS reactor was heated to 5000 under 25 ml/min nitrogen at 50 or 100 psig. After the catalyst temperature was stable, 5 grams/hr of a mixture of 1-methyl naphthalene in 10 volume % hydrogen/nitrogen was introduced to the catalyst in a top/down flow. The liquid product was collected in a single stage separator at 0 psig and 22.5 C and the light gas product from the separator was analyzed on-line using an Agilent 7890B GC equipped with a 100 meter DHA boiling point column and FID. Liquid product from the separator was analyzed off-line by gas chromatography using the same GC configuration. Conversion of 1 methyl naphthalene and yield to BTX are presented in the table below.

| | methyl | | | | 1 methyl | θTX yield as function of |
|---|---|---|---|---|---|---|
| | naphthalene | 10% H2/N2 | | | naphthalene | reacted 1-methyl |
| total WHSV | WHSV | WHSV | psig | Temp C. | reacted | naphthalene |
| 1 | 0.5 | 0.5 | 50 | 500 | 7.7% | 75.4% |
| 1 | 0.5 | 0.5 | 100 | 500 | 17.3% | 56.1% |

1. A process for high-selectivity conversion of a first composition comprising olefins to a second composition comprising at least one monocyclic aromatic compound, comprising the steps of:
a) introducing the first composition to at least one catalyst capable of converting olefins to at least one monocyclic aromatic compound;
b) operating said process using a reactor at high severity to result in the production of at least one monocyclic aromatic compound; and at least one of:
c) introducing at least one weakly coordinating compound to the catalyst; and c2) forming at least one polyaromatic compound in said process, wherein said at least one polyaromatic compound is recycled to a gasifier or pyrolysis reactor or catalytic reactor.

2. The process according to claim 1, further comprising the step of subjecting the first composition with a nitrogen and/or sulfur atom and/or halogen-containing compound removal process.

3. (canceled)

4. The process according to claim 1, wherein said at least one catalyst is porous aluminosilicate or zeolitic material with a portion of its pores in the micro-, meso- and/or macro-range.

5. The process according to claim 1, wherein said at least one catalyst is subject to a regeneration process, wherein said regeneration process comprises the introduction of inert gas and/or an oxidant and/or a reductive fluid.

6. The process according to claim 4, wherein said high severity is in an outlet or inlet of said reactor, or intra-reactor, and further wherein said high severity is greater than 425° C..

7. The process according to claim 6, wherein said weakly-coordinating compound reduces the H—O bond frequency of the zeolite or aluminosilicate framework by about 1 to 300 cm⁻¹ as measured by FT-IR.

8. The process according to claim 1, wherein said olefins have a carbon chain length of C2 to C4.

9. (canceled)

10. (canceled)

11. The process according to claim 1, further comprising the step of removing at least one by-product before introducing the first composition to at least one zeolitic catalyst.

12. The process according to claim 11, wherein said at least one by-product is removed by adsorption, aqueous redox reaction, solvent or solid absorption, electrostatic precipitation, centrifugal separation, or filtration.

13. (canceled)

14. (canceled)

15. The process according to claim 1, wherein said high-selectivity conversion of a first composition comprising olefins to a second composition comprising the monocyclic aromatic compounds is greater than 25.

16. (canceled)

17. The process of claim 1, further comprising: subjecting a feed gas to a compound removal process to yield the first composition, the compound removal process comprising:
inputting the feed gas and a compound removal solution into a compound removal reactor; and
outputting from the reactor an output including the first composition.

18. The process of claim 17, the compound removal solution including:
circulating ammonium polysulfide (APS);
diammonium polysulfide;
ammonium hydroxide; or
a mixture thereof.

19. (canceled)

20. (canceled)

21. The process of claim 17, the compound removal solution including a mixture of a first compound removal solution component and a second compound removal solution component in aqueous solution at a weight ratio of 1:2 at approximately 20-40° C., wherein the second compound removal solution component is an alkaline component.

22. A process for supplying a first composition to a downstream catalytic process, comprising:
inputting the feed gas and a compound removal solution into a compound removal contactor; and
operating the reactor to remove at least one by-product from the feed gas; and, outputting from the reactor an output composition to the downstream catalytic process, the output composition including at least one olefin.

23. The process of claim 22, the compound removal solution including:
circulating ammonium polysulfide (APS);
diammonium polysulfide;
ammonium hydroxide; or
a combination thereof.

24. (canceled)

25. (canceled)

26. The process of claim 22, the compound removal solution including a mixture of a first compound removal solution component and a second compound removal solution component in aqueous solution at a weight ratio of 1:2 at approximately 20-40° C.

27. The process of claim 22, further comprising:
outputting from the compound removal contactor an additional output including a by-product removal output;
stripping at least one component of the by-product removal output in a vessel separate from the reactor.

28. The process of claim 27, the by-product removal output including ammonium polysulfide (APS).

29. The process of claim 28, the at least one component of the by-product removal output including gaseous ammonia and hydrogen sulfide.

30. (canceled)

31. (canceled)

32. The process of claim 27, the at least one component including ammonium polysulfide (APS), the process further comprising circulating the APS to the compound removal contactor.
